(19) United States
(12) Patent Application Publication
(10) Pub. No.: US 2011/0119794 A1
(43) Pub. Date: May 19, 2011

(54) RICE NON-ENDOSPERM TISSUE EXPRESSION PROMOTER (OSTSP 1) AND THE USE THEREOF

(75) Inventors: Jianbo Yang, Anhui (CN); Yuping Lu, Anhui (CN); Ying Wu, Anhui (CN); Meimei Wang, Anhui (CN); Xuzhong Li, Anhui (CN); Fengshun Song, Anhui (CN); Yi Zhang, Anhui (CN); Xiufeng Wang, Anhui (CN); Ni Dahu, Anhui (CN)

(73) Assignee: Syngenta Participations AG, Anhui Academy of Agricultural Sciences (CN)

(21) Appl. No.: 12/741,226

(22) PCT Filed: Nov. 19, 2008
(86) PCT No.: PCT/IB2008/054865

§ 371 Date: Feb. 1, 2011

(30) Foreign Application Priority Data
Nov. 19, 2007 (CN) 200710190007.4

(51) Int. Cl. A01H 5/10; C07H 21/04; C12N 15/63; A01H 5/00; C12N 5/10

(52) U.S. Cl. 800/300; 536/24.1; 435/320.1; 536/24.33; 800/298; 800/301; 800/302; 435/418; 435/419

(57) ABSTRACT

An isolated rice non-endosperm tissue expression promoter, OsTSP I, and the use thereof. The promoter comprises the defined sequence of 1785 by (SEQ ID NO: 1), given in the specification, or its fragment or variant, or a nucleotide sequence If which hybridizes to SEQ ID NO: 1, or its fragment or variant, under stringent conditions. The activity of OsTSP I is comfirmed by transgenic methods. As determined histochemically, OsTSP I reglulates GUS expression in a tissue-specific manner and is not active in endosperm tissues. The OsTSP I can be used as a powerful tool for the investigation and control of gene expression in rice and other crops. It is particularly advantageous for development of safe transgenic foods such as rice.

### FIELD OF THE INVENTION

The present application claims the priority of CN 2007 10190007.4, filed Nov. 19, 2007, the entire content of which is specifically incorporated by reference.

The present disclosure relates generally to a rice non-endosperm tissue promoter and uses thereof. The disclosure further relates to plants transformed with the inventive promoter wherein genes under the control of the disclosed promoter are expressed in non-endosperm tissues.

### BACKGROUND OF THE INVENTION

Genetic engineering technology has led to the development of many transgenic plant species and varieties. Successfully engineered plant species include rice (Oryza sativa).

However, several severe problems may limit the commercialization and utilization of transgenic plants. The commercialization of transgenic rice is hindered by the accumulation of exogenous proteins in endosperm. Such accumulation may engender concerns as to food safety. Expressing the exogenous genes only in non-edible parts of the plant in an effective approach to solving this problem.

By utilizing a gene promoter strategy (i.e., using an inducible, tissue specific or time-dependent promoter), to ensure that the edible parts of the plant do not contain the expression products of the exogenous genes, and accordingly, to reduce the potential risks to human health and to promote the commercialization of transgenic rice.

A promoter is a DNA sequence for localization of RNA polymerases, generally upstream of gene coding regions. Once the RNA polymerase is localized on and bound to the promoter, it can start the transcription of genes. The interaction of the promoter and the RNA polymerase as well as other trans-acting elements, such as protein cofactors, form the core of a gene expression control pattern. Generally, there are cis-acting elements, specific DNA sequences upstream of the promoter, which bind transcription factors to activate or inhibit gene transcription. Promoters are necessary gene expression of genes, and their sequence features and interactions with specific transcription factors determine the spatial and temporal features, the intensity of the expression of the exogenous genes and the like. Known tissue specific promoters include seed specific promoters, fruit specific promoters, stem specific promoters, mesophyll cell specific promoters, root specific promoters, and the like. Some tissue specific promoters which have been used in molecular breeding are listed in Table 1.

TABLE 1
Tissue Specific Promoters Commonly Used in Molecular Breeding

| Tissue | Promoter Source | Gene of Interest | Plant | Author |
|---|---|---|---|---|
| Anther | tobacco Ta29 | Bar | tobacco | Mariani (1990) |
| | | | and cole | |
| Seed | phaseolus vulgaris | α ai (Lectin gene) | tobacco | Altabella (1990) |
| | agglutinin gene | | | |
| | PHA-L | | | |
| Leaf | PEPC | Cry1A(b) | maize | Kozial (1993) |
| | (Pepcarboxylase | | | |
| | Gene of Maize) | | | |
| Phloem | rice sucrose | GNA (Snowdrop Lectin) | tobacco | Shi (1994) |
| | synthase gene | | | |
| | RSs1 | | | |
| Fruit | Ovary tissue | IPT (isopentenyl transferase) | tobacco | Martineau (1995) |
| | (patent) | | | |
| Fruit | tomato 2A12 | IPT (isopentenyl transferase) | tomato | Mao et al. (2002) |
| | promoter | | | |
| endosperm | maize starch | GUS | maize | Russell (1997) |
| | synthase gene | | | |
| | GBS | | | |
| root system | Agrobacterium | ACC (1-amino- | tomato | Varvara (2001) |
| | rhizogenes rolD | cyclopropane-1-carboxylic | | |
| | promoter | acid) deaminase gene | | |
| Core | maize pGL2 | Cry1A(b) | rice | Datta (1998) |

### SUMMARY OF THE INVENTION

The present disclosure provides an isolated nucleic acid molecule (polynucleotide) having a plant nucleotide sequence that directs transcription of a linked nucleic acid segment in a non-endosperm tissue of a plant or plant cell, e.g., a linked plant DNA comprising an open reading frame for a structural or regulatory gene. The nucleotide sequence preferably is obtained or isolatable from plant genomic DNA.

The present disclosure also provides a plant promoter, an isolated nucleic acid molecule having a plant nucleotide sequence, that directs constitutive transcription of a linked nucleic acid segment in non-endosperm tissues of a host cell, e.g., a plant cell. The nucleotide sequence preferably is obtained or isolatable from plant genomic DNA. In particular, the nucleotide sequence is obtained or isolatable from an Oryza sativa gene which directs transcription of a linked nucleic acid segment only in non-endosperm tissues.

The present disclosure further provides an isolated nucleic acid molecule which comprises a plant nucleotide sequence that directs preferential transcription of a linked nucleic acid segment in plant non-endosperm tissues (i.e., root, stem, and leaf).

The present disclosure provides a plant promoter, an isolated nucleic acid molecule, characterized as having the sequence SEQ ID NO: 1. Some aspects of the present disclosure provide the rice, non-endosperm-tissue promoter OsTSP 1.

In certain aspects, the plant nucleotide sequences hybridize under high stringency conditions to a complement of sequence SEQ ID NO: 1. In other aspects, the plant nucleotide sequence is a functional fragment from about 25 to about 2000 nucleotides in length.

The present disclosure provides defined hybridization conditions for SEQ ID NO: 1. Some aspects of the present disclosure provide for high stringency hybridization conditions such as repeated washing for 30 minutes at 65° C. in a solution comprising 2× SSC (300 mM NaCl, 30 mM sodium citrate, pH 7.0) 0.5% (w/v) SDS solution. Some aspects of the present disclosure provide for low stringency hybridization conditions such as repeated washing for 30 minutes at 42° C. in a solution comprising 2× SSC, 0.5% (w/v) SDS solution.

The present disclosure provides a rice, non-endosperm-tissue expression vector comprising a rice, non-endosperm-tissue promoter operably-linked to a gene-of-interest (GOI) downstream of the promoter. Certain aspects of the disclosure provide the rice, non-endosperm-tissue promoter and a gene-of-interest (GOI) are inserted into a plasmid. Preferably, the plasmid is pCAMBIA 1305.1 wherein the cauliflower mosaic virus (CaMV) 35S promoter (CaMV 35S promoter) is replaced with an OsTSP promoter. Preferably, the gene-of-interest is the GUS gene. Non-limiting genes-of-interest may include: an anti-insect gene, an anti-bacterial gene, an anti-fungal gene, an anti-viral gene, an anti-nematode gene, an anti- herbicide gene, an selectable marker gene, a high-yield gene, a high-quality gene, a transcript of polypeptide product, or RNA molecule.

The present disclosure provides an effective approach to promote the commercialization of transgenic rice by expressing exogenous genes in rice non-endosperm tissues, such as leaf and stem. Consumption of exogenous proteins expressed in transgenic rice may confer potential risks to human health. The present disclosure provides a means of limiting the expression of exogenous proteins to non-endosperm tissues, thus reducing or eliminating the risks to human health.

The present disclosure provides a means of protecting a plant against a pest while limiting the exposure to humans. The protecting means may be the expression of Bacillus thuringiensis (BT) toxic protein in rice trophosome, but not endosperm tissues.

Still other aspects and advantages of the present invention will become readily apparent by those skilled in the art from the following detailed description The description is to be regarded as illustrative in nature and not as restrictive.

### BRIEF DESCRIPTION OF THE DRAWING

The invention is best understood from the following detailed description when read in connection with the accompanying drawing. Included in the drawing are the following figures:

FIG. 1 shows the sequences of the promoter OsTSP I and the PCR primers for amplification. FIG. 1A shows the sequence of the promoter OsTSP I (SEQ ID NO: 4) containing the PCR primer sequences and has a length of 1811 base pairs. EcoRI site: 8-13, and BamHI site: 1799-1804. FIG. 1B shows the PCR forward primer OsTSP I-F (SEQ ID NO: 2) (containing the EcoRI site) of the promoter OsTSP I. FIG. 1C shows the PCR reverse primer OsTSP I-R (SEQ ID NO: 3 (containing the BamHI site) of the promoter OsTSP I.

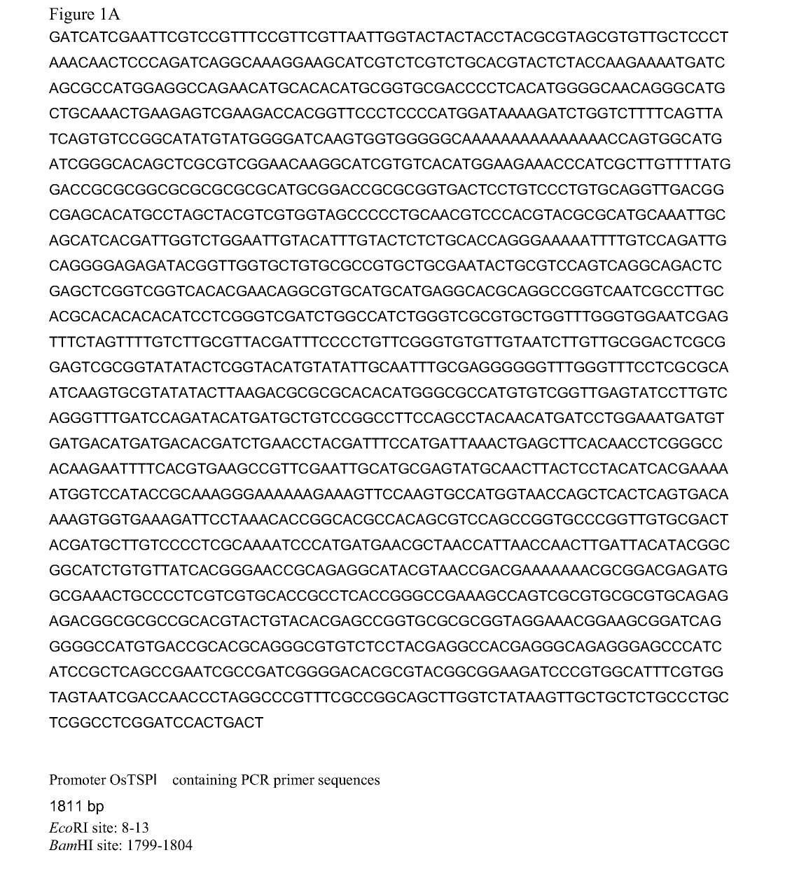

FIG. 2 is a cloning schematic to confirm the function of promoter OsTSP I.

FIG. 3 shows the electrophoretogram of RT-PCR products from rice tissues. (a) Results of total RNA extraction from various tissues of rice show that the extracted total RNA can be used in the experiments, since there appears the clear 18S and 28S bands. (b) PCR results of the internal standard gene β-actin in RNA from various tissues of rice treated with DNase I, show that DNA was totally removed from the RNA sample for the absence of β-actin gene band (158 bp) in the results of six tissues tested. (c) RT-PCR results of the internal standard gene β-actin in the rice tissues show that the internal standard gene can be found in all tissues of rice. (d) RT-PCR results of the amplified product (143bp) of the gene (GI21104672) driven by the candidate promoter in rice tissues show that the gene driven by the promoter was expressed in tissues other than the endosperm during grain filling period. Lanes: M: 100 bp DNA Marker, 1: root, 2: stem, 3: leaf, 4: flower, 5: glume, 6: endosperm during grain filing period (10-15 days after anthesis), and 7: DNA control.

FIG. 4 shows the electrophoretogram of PCR products of the promoter OsTSP I. Using extracted Nipponbare (Gramene.org accession name for Oryza sativa japonica germplasm) DNA as the template, the candidate promoter was cloned with the primers given in FIG. 1B and FIG. 1C. Lanes: M: DL2000 DNA Marker, and 1: PCR amplification product of about 1800 bp.

FIG. 5 shows the structure of T-DNA region of non-endosperm tissue specific expression vector pOsTSP I-GUS. The CaMV35S promoter was excised from vector pCAMBIA1305.1 using restriction enzymes HindIII and NcoI. After blunting and ligating the ends with ligase, a intermediate vector pCAMBIA1305.1(−) was generated. Then, pGEM-OsTSP I was double-digested with EcoRI and BamHI to produce the OsTSP I fragment. The resulted OsTSP I fragment was recombined with the vector pCAMBIA1305.1(−) which had also been digested with EcoRI and BamHI to obtain the expression vector pOsTSP I-GUS. The structure of T-DNA region of pOsTSP I-GUS is shown, P_{35S}: CaMV35S promoter, T_{35S}: CaMV terminator, Hyg: hygromycin resistance gene, and T_{NOS}: NOS terminator.

FIG. 6 shows PCR identification and confirmation of the transgenic rice plants. A: The 35S fragment can be amplified from the 35S-GUS transgenic rice plants but not from non-transgenic rice plants. B: The GUS fragment can be amplified from the 35S-GUS transgenic rice plants but not from non-transgenic rice plants. C: The GUS fragment can be amplified from the OsTSP I-GUS transgenic rice plants but not from non-transgenic rice plants. Lanes: M1: 50 bp DNA Marker, M2: DL2000 DNA Marker, P1: pCAMBIA1305.1 (35S-GUS), P2: pOsTSP I-GUS, CK(−): non-transgenic control, and 1-20: transgenic plants.

FIG. 7 shows the GUS histochemical staining results of leaves of T₀ generation and endosperm of progeny seeds of positive transgenic rice. The blue staining can be found both in leaves and endosperm of the 35S-GUS transgenic rice, and can also be found in leaves but not in endosperm of the OsTSP I-GUS transgenic rice. These results demonstrate the tissue-specific expression of the GUS gene driven by the OsTSP I, i.e., non-endosperm expression specificity. A: GUS histochemical staining results of leaves of T₀ generation and endosperm of progeny seeds of the 35S-GUS transgenic rice; B: GUS histochemical staining results of leaves of T₀ generation and endosperm of progeny seeds of the OsTSP I-GUS transgenic rice; and C: non-transgenic plants as the negative control. I: leaves; and II: endosperm of mature seeds.

### DEFINITIONS

In describing the present invention, the following terms will be employed, and are intended to be defined as indicated below.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA or functional RNA, or encodes a specific protein, and which includes regulatory sequences. Genes also include nonexpressed DNA segments that, for example, form recognition sequences for other proteins. A gene may also comprise other 5′ and 3′ untranslated sequences and termination sequences. Further elements that may be present are, for example, introns. Genes can be obtained from a variety of sources, including by cloning from a source of interest or by synthesis using a known or predicted sequence, and may include sequences designed to have desired parameters.

A “marker gene” encodes a selectable or screenable trait.

Chimeric Gene: A recombinant DNA sequence in which a promoter or regulatory DNA sequence is operatively linked to, or associated with, a DNA sequence that codes for an mRNA or which is expressed as a protein, such that the regulator DNA sequence is able to regulate transcription or expression of the associated DNA sequence. The regulator DNA sequence of the chimeric gene is not normally operatively linked to the associated DNA sequence as found in nature.

Associated With/Operatively Linked: Refers to two DNA sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

Coding Sequence: a nucleic acid sequence that is transcribed into RNA such as mRNA, rRNA, tRNA, snRNA, sense RNA or antisense RNA. Preferably the RNA is then translated in an organism to produce a protein.

Complementary: refers to two nucleotide sequences that comprise antiparallel nucleotide sequences capable of pairing with one another upon formation of hydrogen bonds between the complementary base residues in the antiparallel nucleotide sequences.

Expression: refers to the transcription and/or translation of an endogenous gene or a transgene in plants. In the case of antisense constructs, for example, expression may refer to the transcription of the antisense DNA only.

Expression Cassette: A nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue, or organ, or stage of development.

Heterologous DNA Sequence: The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also includes non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

Homologous DNA Sequence: A DNA sequence naturally associated with a host cell into which it is introduced.

Isocoding: A nucleic acid sequence is isocoding with a reference nucleic acid sequence when the nucleic acid sequence encodes a polypeptide having the same amino acid sequence as the polypeptide encoded by the reference nucleic acid sequence.

Isolated: In the context of the present invention, an isolated nucleic acid molecule or an isolated enzyme is a nucleic acid molecule or enzyme that, by the hand of man, exists apart from its native environment and is therefore not a product of nature. An isolated nucleic acid molecule or enzyme may exist in a purified form or may exist in a non-native environment such as, for example, a recombinant host cell.

Native: refers to a gene that is present in the genome of an untransformed cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

The GUS gene encodes GUS encodes β-glucuronidase (E.C. 3.2.1.31) which catalyzes the hydrolysis of a wide variety of natural and synthetic β-glucuronides. Artificial substrates include: p-nitrophenyl glucuronide, 4-methyl umbelliferyl glucuronide, and 5-bromo-4-chloro-3-indolyl glucuronide (X-Gluc).

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (191); Ohtsuka et al., J. iol. Chem. 260: 2605-2608 (1985); Rossolini et al., Mol. Cell. Probes 8: 91-98 (1994)). The terms “nucleic acid” or “nucleic acid sequence” may also be used interchangeably with gene, cDNA, and mRNA encoded by a gene. In the context of the present invention, the nucleic acid molecule is preferably a segment of DNA. Nucleotides are indicated by their bases by the following standard abbreviations: adenine (A), cytosine (C), thymine (T), and guanine (G).

The terms “open reading frame” and “ORF” refer to the amino acid sequence encoded between translation initiation and termination codons of a coding sequence. The terms “initiation codon” and “termination codon” refer to a unit of three adjacent nucleotides ('codon') in a coding sequence that specifies initiation and chain termination, respectively, of protein synthesis (mRNA translation).

Plant: Any whole plant.

Plant Cell: Structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

Plant Cell Culture: Cultures of plant units such as, for example, protoplasts, cell culture cells, cells in plant tissues, pollen, pollen tubes, ovules, embryo sacs, zygotes and embryos at various stages of development.

Plant Material: Refers to leaves, stems, roots, flowers or flower parts, fruits, pollen, egg cells, zygotes, seeds, cuttings, cell or tissue cultures, or any other part or product of a plant.

Plant Organ: A distinct and visibly structured and differentiated part of a plant such as a root, stem, leaf, flower bud, or embryo.

Plant tissue: A group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

Minimal Promoter: a promoter element, particularly a TATA element, that is inactive or has greatly reduced promoter activity in the absence of upstream activation. In the presence of a suitable transcription factor, a minimal promoter functions to permit transcription.

Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

Vector” is defined to include, inter alia, any plasmid, cosmid, phage or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

A plant transformation vector comprises one or more DNA vectors for achieving plant transformation. For example, it is a common practice in the art to utilize plant transformation vectors that comprise more than one contiguous DNA segment. These vectors are often referred to in the art as binary vectors.

Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a polynucleotide of interest (i.e., a polynucleotide engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens et al., 2000). Several types of Agrobacterium strains (e.g., LBA4404, GV3101, EHA101, EHA105, etc.) can be used for plant transformation. The second plasmid vector is not necessary for introduction of polynucleotides into plants by other methods such as microprojection, microinjection, electroporation, polyethylene glycol, etc.

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

Constitutive expression” refers to expression using a constitutive or regulated promoter. “Conditional” and “regulated expression” refer to expression controlled by a regulated promoter.

Constitutive promoter” refers to a promoter that is able to express the open reading frame (ORF) that it controls in all or nearly all of the plant tissues during all or nearly all developmental stages of the plant. Each of the transcription-activating elements do not exhibit an absolute tissue-specificity, but mediate transcriptional activation in most plant parts at a level of .gtoreq.1% of the level reached in the part of the plant in which transcription is most active.

Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989). Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysome-inducible systems.

Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

Inducible promoter” refers to those regulated promoters that can be turned on in one or more cell types by an external stimulus, such as a chemical, light, hormone, stress, or a pathogen.

Protoplast: An isolated plant cell without a cell wall or with only parts of the cell wall.

Purified: the term “purified,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

Recombinant DNA molecule: a combination of DNA molecules that are joined together using recombinant DNA technology.

Regulatory Elements: Sequences involved in controlling the expression of a nucleotide sequence. Regulatory elements comprise a promoter operably linked to the nucleotide sequence of interest and termination signals. They also typically encompass sequences required for proper translation of the nucleotide sequence.

Selectable marker gene: a gene whose expression in a plant cell gives the cell a selective advantage. The selective advantage possessed by the cells transformed with the selectable marker gene may be due to their ability to grow in the presence of a negative selective agent, such as an antibiotic or a herbicide, compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a plant cell gives the cell both, a negative and a positive selective advantage.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

Substantially identical: the phrase “substantially identical,” in the context of two nucleic acid or protein sequences, refers to two or more sequences or subsequences that have at least 60%, preferably 80%, more preferably 90-95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In one aspect, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.go- v/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1.times.SSC at 45.degree.C. for 15 minutes. An example low stringency wash for a duplex of, e.g., more than 100 nucleotides, is 4-6.times.SSC at 40.degree.C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30.degree. C. Stringent conditions can also be achieved with the addition of destabilizing agents such as formamide. In general, a signal to noise ratio of 2.times. (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

A “subsequence” refers to a sequence of nucleic acids or amino acids that comprise a part of a longer sequence of nucleic acids or amino acids (e.g., protein) respectively.

Nucleic acids are “elongated” when additional nucleotides (or other analogous molecules) are incorporated into the nucleic acid. Most commonly, this is performed with a polymerase (e.g., a DNA polymerase), e.g., a polymerase which adds sequences at the 3′ terminus of the nucleic acid.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

Transformation: a process for introducing heterologous DNA into a host cell or organism.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

### DETAILED DESCRIPTION

Reference is made to the figures to illustrate the following examples. It is to be understood that the invention is not hereby limited to those aspects depicted in the figures.

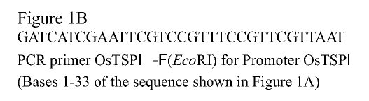

The invention is illustrated more specifically by incorporating the following examples. In the examples, the rice plants were transformed by the GUS gene driven by the promoter OsTSP I. The scheme of the invention is shown in FIG. 2.

1. Isolation and Sequence Analysis of the Promoter OsTSP I.

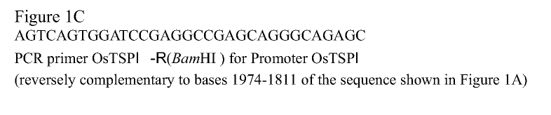

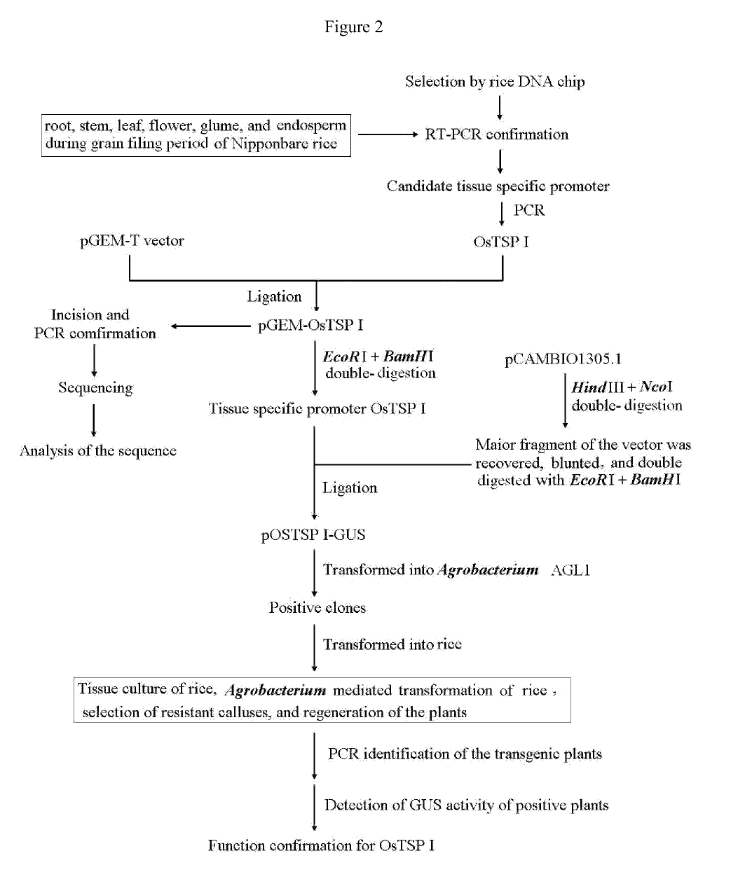

Promoter-dependent gene expression profiles in root, stem, leaf, flower, glume, and endosperm tissues during the rice grain-filling period (10-15 days after anthesis) were analyzed using rice genome chips and were confirmed by RT-PCR (FIG. 3). Initially, we found OsTSP I, a rice non-endosperm tissue expression promoter which was amplified by PCR (FIG. 4). PCR-amplified products were cloned into pGEM-T (Promega, Madison, Wis.) and a length of 1785 by was determined by sequencing. OsTSP I-positive clones were named pGEM-OsTSP I. Neural Network Promoter Prediction online software was used to predict the core sequence and transcriptional start site of the promoter. The OsTSP I core sequence is most likely to be in positions 45 bp-95 bp, 849 bp-899 bp, 920 bp-970 bp, and 1423 bp-1473 bp, with the probability of 0.80, 0.86, 0.97, and 0.99, respectively. The general features eukaryotic promoters suggest that the OsTSP I transcriptional start site (i.e. the cap structure) is the A in position 85 before ATG. Sequence analysis using promoter predictive software PLACE demonstrates that OsTSP I contains numerous cis-acting elements. The main regulatory elements of OsTSP I are shown in table 2.

TABLE 2
Structure Analysis of the OsTSP I Sequence.

| Name of the | | | |
|---|---|---|---|
| transcription | | | |
| regulator or site | Localization | Base sequence | No. |
| TATA-box | (−) 25 | TATA | S000109 |
| ACGTABOX | (+) 1420 | TACGTA | S000130 |
| | (−)1420 | | |
| CAATBOX1 | (+) 704, 883, 915 | CAAT | S000028 |
| | (−) 19, 513, 527, 538, | | |
| | 582, 879, 1146 | | |
| CACGTGMOTIF | (+) 1129 | CACGTG | S000042 |
| | (−) 1129 | | |
| CCAATBOX1 | (−) 19, 527 | CCAAT | S000030 |
| GATA-box | (+) 229, 595, 997 | GATA | S000039 |
| | (−) 253, 974, 1398 | | |
| IBOXCORE | (+) 229 | GATAA | S000199 |
| | (−) 252, 1397 | | |
| GT1CORE | 1363 (−) | GGTTAA | S000125 |

2. Construction of Rice Non-Endosperm Tissue Expression Vector pOsTSP I-GUS.

Plasmid pCAMBIA 1305.1 (CAMBIA.ORG) was modified by excising the

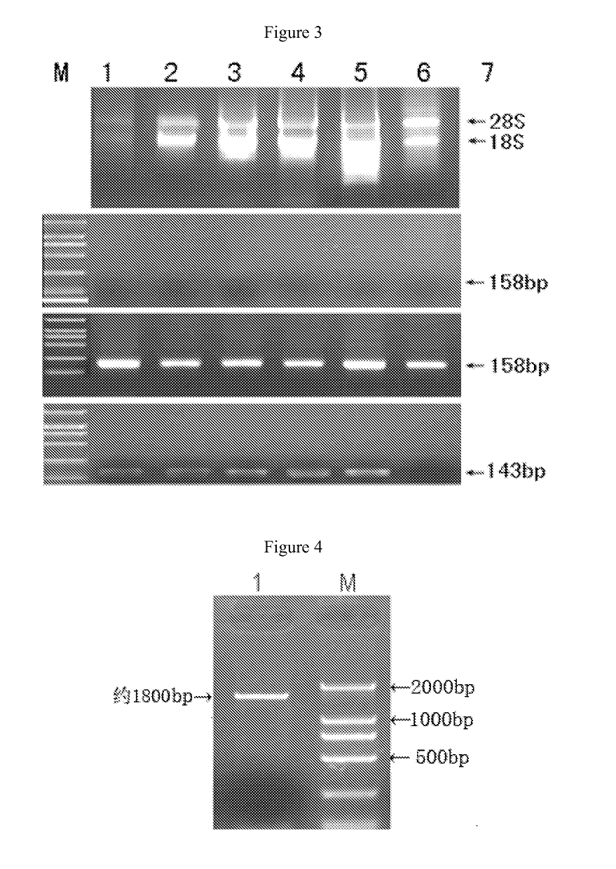

CaMV35S promoter with restriction enzymes HindiIII and NcoI, blunting, and ligating the ends with ligase, which generated a intermediate vector pCAMBIA 1305.1(−) which was then restricted with EcoRI and BamHI. Plasmid pGEM-OsTSP I was double-digested with EcoRI and BamHI to produce an OsTSP I fragment which was recombined with the EcoRI and BamHI-restricted pCAMBIA 1305.1(−) to obtain the expression vector pOsTSP I-GUS. The structure of T-DNA region of expression vector pOsTSP I-GUS is shown in FIG. 5.

3. Agrobacterium tumefaciens-mediated transformation of rice.

Immature embryo of Nipponbare rice (“Nipponbare” is the Gramene.Org accession name for the rice variety Oryza sativa japonica) were induced to form primary calluses about twelve days anthesis and used as the recipient material after two passages. Recombination pOsTSP I-GUS vectors, constructed as above, were introduced into Agrobacterium tumefaciens AGL1 by freeze-thaw and co-cultured with Nipponbare recipients. Transformants were selected. Nipponbare transformed by native pCAMBIA 1305.1 (35S-GUS) were used as positive controls, and non-transformed Nipponbare were negative controls.

4. PCR Identification of Transgenic Plants.

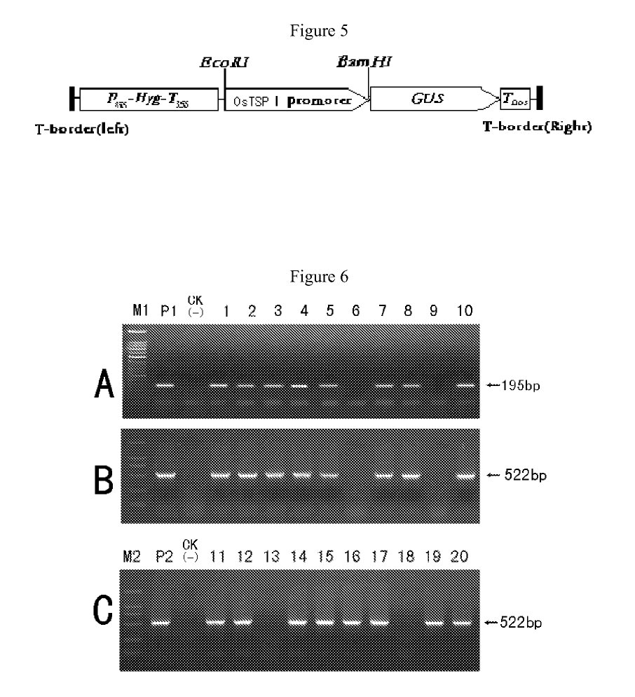

DNA was extracted from leaves and used as templates to detect GUS gene by PCR (FIG. 6).

5. Functional Characterization of Promoter OsTSP I.

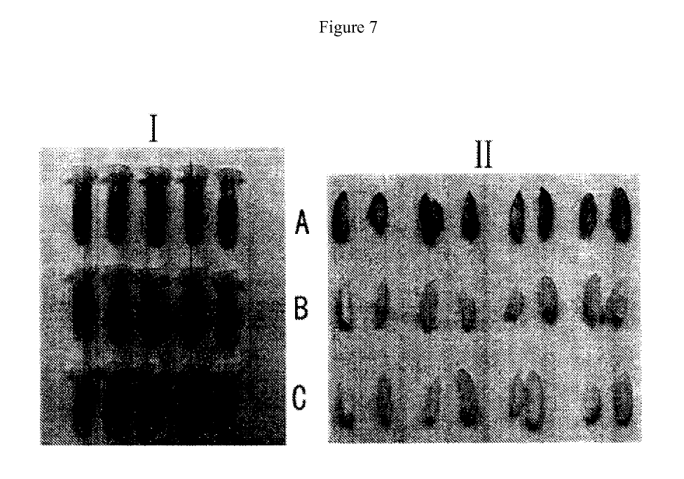

PCR-positive transgenic plants were field-grown. Various tissues and organs, at several developmental stages, from 15 sample plants were histochemically-stained for GUS activity to characterize the tissue specificity of reporter-gene expression (Table 3 and FIG. 7). Blue-stained tissues were positive for the GUS-activity, (+); non-staining tissues were negative for GUS-activity (−).

TABLE 3
GUS Histochemical staining in Transgenic Rice.

| Vector | | pCAMBIA | | |
|---|---|---|---|---|
| Tissues or organs | GUS activity | 1305.1 | pOsTSP I-GUS | CK (−) |
| root | (+) | 15 | 15 | 0 |
| | (−) | 0 | 0 | 15 |
| stem | (+) | 15 | 15 | 0 |
| | (−) | 0 | 0 | 15 |
| leaf | (+) | 15 | 15 | 0 |
| | (−) | 0 | 0 | 15 |
| immature | (+) | 15 | 0 | 0 |
| endosperm | (−) | 0 | 15 | 15 |
| mature | (+) | 15 | 0 | 0 |
| endosperm | (−) | 0 | 15 | 15 |

6. Selection of a Tissue Specific Expression Promoter.

6.1 Primer design. Primers were designed using the amplification conditions set forth below. The primers were synthesized by Shanghai Biological Engineering Corporation.

6.2 Sequences of the primers and the amplification conditions. Gene GI21104672 was expressed using the OsTSP I promoter. The gene was amplified with a forward primer 5′-GAACAGTCCAGCAGCGTAA-3′ (SEQ ID NO: 2), a reverse primer 5′-CCACAGCCCACCATCATAC-3′ (SEQ ID NO: 3) and temperature cycling conditions: 94° C. 5 mins, 94° C. 30 sec, 60° C. 30 sec, 72° C. 30 sec, 35 Cycles 72° C. 7 mins.

The internal β-actin gene (158 bp) was amplified with a forward primer 5′-TATGGTCAAGGCTGGGTTCG-3′ (SEQ ID NO: 7), a reverse primer 5′-CCATGCTCGATGGGGTACTT-3′ (SEQ ID NO: 8) and temperature cycling conditions: 94° C. 5 mins, 94° C. 30 sec, 60° C. 30 sec, 72° C. 30 sec, 35 Cycles 72° C. 7 mins.

The OsTSP I promoter (1785 bp) was amplified with a forward primer OsTSPI-F(EcoRI): 5′-GATCATCGAATTCGTCCGTTTCCGTTCGTTAAT-3′ (SEQ ID NO: 2), a reverse primer OsTSPI-R(BamHI): 5′-AGTCAGTGGATCCGAGGCCGAGCAGGGCAGAGC-3′ (SEQ ID NO: 3) and temperature cycling conditions: 94° C. 5 mins, 94° C. 1 min, 56° C. 1 min, 72° C. 1.5 min, 35 Cycles 72° C. 7 mins.

6.3 Extraction of total RNA from rice tissues. Various tissues, including: root, stem, leaf, flower, glume, and endosperm were extracted for total RNA present during the grain filling period of normally growing rice. Extraction was performed by column chromatography using a ^{UNIQ}10™ Total RNA Extraction and Purification Kit (Sangon, Shanghai, CN).

Samples were lysed after trituration in liquid Nitrogen. Samples (up to 100 mg) were triturated in liquid nitrogen. The nitrogen was evaporated from the resulted powders after transfer to 1.5 ml centrifuge tubes ensuring the sample did not thaw. RLT (RLT is a component of the UNIQ-10™ kit) solution (450 μL) was added to the sample, and the mixture was mixed by intensive shaking and allowed to lyse by standing at 56° C. for 1-3 minutes. Preferably, samples with a high starch content should be lysed at low temperatures, otherwise an agglomerated mass may form.

RNA was freed of contaminants using a UNIQ-10™ column. A portion of thawed sample was mixed with 0.5 volume of absolute alcohol. A 700 μL portion of the sample, possibly containing precipitates, was loaded on the UNIQ-10™ column placed in a 2 mL recovering tube and centrifuged at 8,000×g for 1 min. The eluate was discarded. The column was loaded with 500 μL RW Solution (a component of the UNIQ-10™ kit), allowed to stand at room temperature for 1 min, and centrifuged at 10,000×g for 30 seconds.

The column was washed twice with 500 μL, portions of RPE Solution (a component of the UNIQ-10™ kit) by centrifugation at 10000×g for 30 seconds. The eluates were discarded. The column was freed of residual RPE solution by centrifugation at 10000×g for 15 seconds.

The UNIQ-10™ columns were transferred to 1.5 mL, sterilized, RNase-free centrifuge tubes. DEPC-H₂O (30-50 μL) (a component of the UNIQ-10™ kit) was added to the center of the column membrane and the columns were incubated at 50° C. for 2 minutes. RNA was eluted by centrifugation at 8,000×g for 1 min. The eluted RNA may be used immediately or stored at −20° C. or lower for later use. The quality of the extracted RNA was evaluated by electrophoresis.

6.4 DNAase I digestion of DNA. RNA was reacted on ice for 10-30 minutes in a reaction system comprising (in μl): RNA, 5; DEPC-H₂O, 3.5; 10×DNase I Buffer 1 (400 mM TrisCl (pH 7.5 at 25° C.) , 80 mM MgCl2 , 50 mM DTT); and DNase I (10 U/μL), 0.5.

6.5 Synthesis of the first strand of cDNA. A cDNA synthesis mixture comprising (in μL): 5 total extracted RNA, 5; 10 mmol/L dNTPs, 1; 0.5 μg/μL Oligo(dT) 16, 1 and sufficient DEPC-H₂O to bring the final volume to 10 μL, was reacted in an RNAase-free Eppendorf™ (EP) tube. The mixture was incubated at 65° C. for 5 minutes, and then placed on ice for 1 minute. The cDNA reaction was supplemented with 2 μL 10×buffer (Universal RiboClone® cDNA Synthesis System, Promega, Madison, Wis.), 4 μL 25 mmol/L MgCl₂, 2 μL 0.1 mol/L DTT, and 4 μL RNase-free recombinant RNasin® Ribonuclease Inhibitor (Promega) were added. After mixing, the mixture was centrifuged and heated in a water bath at 42° C. for 2 minutes and supplemented with 1 μL reverse transcriptase (200 Unit/μL, A-MLV, Promega, Madison, Wis.). The reaction was mixed and heated in a water bath at 42° C. for 50 minutes and then at 70° C. for 15 minutes. The resulted product was stored at −20° C. until use.

6.6 PCR amplification of reverse transcription product. The resultant cDNA was amplified by PCR using the rice β-actin gene as an internal standard. The following components were sequentially added to a 0.2 ml EP tube: 10×PCR I Buffer (100 mM Tris-HCl, pH 8.3 at 25° C.; 500 mM KCl; 0.01% gelatin), 2.5 μL; MgCl₂ (25 mmol/L), 2.0 μL; dNTP (2.0 mmol/L), 2.0 μL; Primer-F (6.25 μmol) [SEQ ID NO: 7], 1 μL; Primer-R (6.25 μmol/L)) [SEQ ID NO: 8], 1 μL; Taq enzyme (5 U/μL) (Shanghai Biological Engineering Corp., China), 0.2 μL; template DNA (5 ng/μl ), 2.0 μL; and ddH₂O, 14.3 μL to bring the total volume to 25 μL. The tube contents were mixed amplified under the conditions set forth above. The PCR products (2 μL) were mixed with 2 μL loading buffer (30% Glycerol, 0.025% Bromophenol blue). The fragment length of the amplified products was characterized by electrophoresis on 2% agarose gels at 5V/cm. At least one lane contained marker DNA to serve as a size comparison. Electrophoretograms were visualized with a gel imaging system. The expression profiles of the various rice promoters was established by comparing the sizes of DNA expressed under their control.

7. Cloning and Sequencing of the Promoter OsTSP I.

7.1 Extraction and detection of rice genome. Extraction of rice genome DNA was performed by a modified SDS method. About 100mg leaves were weighed into a 2.0mL centrifuge tube. Liquid nitrogen was added, and the sample was triturated into powders with a glass rod. The powder was extracted with 700 μL SDS extraction buffer (100 mmol/L Tris-HCl (pH 8.0), 20 mmol/L EDTA (pH 8.0), 500 mmol/L NaCl, 1.5% (w/V) SDS for 1 hour in a 60° C. water bath. Chloroform/isoamyl alcohol (24:1) at volume ratio of 1:1 was added, and the mixture was kept at room temperature for 30 minutes. Following centrifugation at 12,000×g for 10 minutes at 4° C., the supernatant was transferred into a clean 1.5 mL centrifuge tube, a 0.6 volume of isopropanol was added. Following a 30 minute incubation at 4° C., the supernatant was discarded after centrifugation at 12,000×g for 10 minutes at ° C. The pellet was washed twice with 70% alcohol by centrifugation at 10,000×g for 5 minutes at 4° C. The precipitates were dissolved in TE (10 mM Tris-HCl, 1 mM EDTA, pH 8.0) and 2 μL RNase was added. The TE solution was incubated with 2 μL RNAase (10 mg/ml) for 30 minutes in a 37° C. water bath and then stored at −20° C. until use.

7.2 Determination of the quality and concentration of the extracted rice genome DNA. DNA solution (2 μL) was electrophoresed on 0.8% agarose gels. The extracted DNA had an OD260/OD280 ratio of 1.8 and a concentration of 300 ng/μL.

7.3 PCR Amplification, Detection, and Recovery of the Promoter OsTSP I Gene.

7.3.1 PCR amplification and detection. The following components were sequentially added into a 0.2 mL PCR tube (in μL): 10×PCR buffer (100 mM Tris-HCl, pH 8.3 at 25° C.; 500 mM KCl; 0.01% gelatin) (2.5), MgCl₂(25 mmol/L) (2.0), dNTP(2.0 mmol/L) (2.5), OsTSP 1 Primer-F (6.25 μmol/L) [SEQ ID NO: 2 (1), OsTSP 1 Primer-R (6.25 μmol/L) [SEQ ID NO: 3] (1), LA Taq enzyme (5 U/μL) (0.4), Template DNA (2), ddH2O (13.6) in a total volume of 25 μL. The amplification procedure was as given above. The amplified products were electrophoresed and compared to standard DNA markers. Where the size of amplified products had expected value, the synthesized DNA was recovered from the corresponding band.

7.3.2 Recovery of the PCR amplified products. Bands containing the DNA of interest were cut from the gel with a scalpel and put into a 1.5 mL centrifuge tube with 400 μL Binding Buffer (from UNIQ-10 kit, Sangon, Shanghai, China) per 100 mg agarose gel (or 100 μL DNA solution). Tubes were incubated in a 50-60° C. water bath with intermittent shaking for 10 minutes until the gel was totally dissolved. The dissolved gel solution was transferred to a UNIQ-10™ column (Sangon, Shanghai) equipped with a 2 mL recovering tube, chilled to −20° C. for 2 minutes, centrifuged at 8,000×g (room temperature) for 1 minute, and the eluate discarded. Columns were washed twice with 500 μL aliquots of Wash Solution (UNIQ-10 kit) by centrifugation at 8,000×g (room temperature) for 1 minute and a final centrifugation (12,000×g for 15 seconds. The wash eluates were discarded. Columns were placed into clean 1.5 mL tubes. The columns were equilibrated for 2 minutes at room temperature with 30 μL Elution Buffer (UNIQ-10 kit) or ddH₂O (pH>7.0). DNA was eluted by centrifugation at 12,000×g for 1 minute. The product was stored frozen at −2° C.

7.3. Construction, sequencing, and analysis of a TA cloning vector containing the OsTSP I promoter gene.

7.3.1. Ligation of products of the promoter OsTSP gene and T vector. A 10 μL reaction mixture comprising 2×Rapid Ligation Buffer (pGEM®-T Vector System kit, Promega), 5 μL; OsTSP PCR products, 3 μL; T4 DNA Ligase (3U/μL), 1 μL; and pGEM®-T vector, 1 μL was centrifuged at 4,000 rpm for 5 sec, left at room temperature for 5 min, kept on ice for 5 min, and stored in a −20° C. freezer.

7.3.2. Preparation of competent E. coli. A single clone was picked, inoculated into 100mL LB (tryptone 10 g/L, yeast extract 5 g/L, NaCl, 10 g/L) liquid medium and cultured overnight at 37° C. with shaking at 200 rpm. A 10 mL aliquot was inoculated into 100 mL LB liquid medium and cultured at 37° C. for 2-3 hours with shaking at 200 rpm, until the culture reached an optical density (600 nm) of 0.3-0.4. The culture was chilled on ice for 20 minutes, centrifuged at 4,000×g for 5 minuets at 4° C., the supernatant discarded. Pellets were resuspended in 30 mL 0.1M ice-cold CaCl₂, and incubated on ice for 30 minutes. Pellets were harvested after centrifugation at 4,000×g for 5 minutes at ° C. and resuspended in 3 mL 0.1M CaCl₂. The suspension was incubated on ice for 4-10 hours, divided into 200 μL-aliquots, and stored at −70° C. for later use or at 4° C. for more immediate use within one week.

7.3.3. Transformation and selection. A 10 μL aliquot of the ligation reaction product was added to 100 μL suspension of competent cells and chilled on ice for 30 minutes. The suspension was heat-shocked in a 42° C. water bath for 90 seconds and immediately transferred to ice for 3-5 minutes. Normal growth of bacteria expressing plasmid-encoded kanamycin-resistance was achieved by supplementing the cell suspension with a 1 ml aliquot of LB liquid medium (Kan-free) and culturing at 37° C. for 1 hr with shaking. Cultures were centrifuged at 10,000×g for 1 minute and 100 μL of the supernatant was plated on a dish containing kanamycin (50 m/mL). The dish was kept facing-upward for 30 minutes to achieve complete absorption of the bacteria by the medium. The dish was inverted and cultured at 37° C. for 16-24 hours.

7.3.4. Identification of Recombinant Plasmids.

PCR detection of the clones. A PCR reaction mixture was prepared as given above in a 0.2 mL PCR tube. A single clone was picked with a sterilized toothpick and mixed with the PCR reaction mixture. The gene for the OsTSP I promoter was determined by PCR as detailed above.

Enzymatic identification of the recombinant plasmid. A positive DNA clone was identified by enzymatic methods using the restriction enzymes EcoR1 and BamH1. Restriction digests were performed at 37° C. overnight in a buffer comprising (in μL): pGEM-OsTSP□, 15; BamH1, 1; EcoR1,1; 10×buffer K (Fermentas Company), 5; and distilled, deionized water. The products were checked by electrophoresis on 0.8% agarose gels and stored at 4° C. The recombinant plasmid was named pGEM-OsTSP I. Positive clones were sequenced.

7.4. Homology search of the sequences and analysis of cis-acting elements. Sequence homology alignments were performed using internet software (BLASTn, National Center for Biotechnology). The OsTSP1 cis-acting elements were analyzed using Plant CARE software on the Fruitfly.org website and PLACE software on the DNA.Affrc website

8. Construction of GUS Plant Expression Vector.

8.1 Preparation of the recombinant plasmid. Plasmid pCAMBIA 1305.1 was double-excised by restricting overnight at 37° C. with HindIII and NcoI to remove the endogenous CaMV35S promoter which regulates GUS. Restriction was performed in a buffer comprising (in μL): pCAMBIA 1305.1, 15; HindIII, 1; NcoI, 1; 10×buffer K, 5; and distilled, deionized water, 28 (total volume 50 μL. Incised plasmids were blunted and circularized by treatment with ligase for 20 minutes at 12° C. in 20 μL of a buffer comprising (in μL): pCAMBIA 1305.1 (HindIII/NcoI), 13; 10×T4 DNA Polymerase Buffer (Promega), 2; 10% bovine serum albumin (BSA), 2; 2 mM dNTPs, 2; and T4 DNA Polymerase, 1. Polymerase was inactivated at 75° C. Plasmids pCAMBIA 1305.1(−) and pGEM-OsTSP1 were double-excised, at 37° C. overnight, with EcoRI and BamHI, respectively, in 50 μL of a buffer comprising (in μL): pGEM-OsTSP□ or pCAMBIA 1305.1(−), 15; EcoR1, 1; BamH1, 1; 10×buffer K, 5; and distilled, deionized water, 28. The fragments of interest were respectively recovered and ligated overnight at 16° C. by T4 DNA ligase in 20 μL of a buffer comprising (in μL): pCAMBIA 1305.1(−) /EcoR1+BamH1, 8.5; pGEM-OsTSP□/EcoR1+BamH1, 8.5; 10×Ligase Buffer, 2; and T4 DNA Ligase, 1. The ligated fragments were transformed into E.coli JM109. The preparation and transformation of competent E coli. cells was as described above.

8.2 Identification of the recombinant plasmid. PCR cloning was performed as described above. Positive clones were transferred into LB liquid medium containing 50 m/ml kanamycin and shake cultured at 37° C. overnight. Plasmids were extracted and confirmed by enzymatic digestion and sequencing. The target recombinant plasmid was named as pOsTSP I-GUS.

8.3 Transformation of Agrobacterium by the Recombinant Plasmid.

8.3.1 Preparation of competent Agrobacterium AGL1 cells. Single Agrobacterium AGL1 clones were inoculated in 5 mL YEP (yeast extract, 10 g/l; peptone, 10 g/l; sodium chloride, 5 g/l; pH 7.0) medium containing corresponding antibiotics and cultured at 28° C. overnight with 200 rpm shaking. A 2 ml aliquot was transferred into 50 ml of YEP liquid medium and cultured at 28° C. with 200 rpm shaking until the OD₆₀₀ reached 0.5-1.0. Cultures were transferred into a sterilized centrifuge tubes and kept on ice for 30 minutes. Cells were harvested by centrifugation at 5,000×g for 5 min at 4° . Cell pellets were resuspended in 1 mL of 20 mmol/L ice-cold CaCl₂ solution. Competent cells can be used immediately or be stored as 200 μl aliquots in sterilized Eppendorf tubes at 4° C. for use within 48 hours.

8.3.2 Transformation of Agrobacterium AGL1. Competent Agrobacterium AGL1 cells were briefly centrifuged and kept on ice. Recombinant pOsTSP□-GUS plasmid (1 ng) was added to 100 μl of competent cells. Plasmids and competent cells were gently mixed and then kept on ice for 30 minutes. The mixture was frozen in liquid nitrogen for 5 minutes then thawed in a 37° C. water bath for 5 minutes. LB liquid medium (900 μl) was added and the mixture was shaken at 200 rpm at 28° C. for 4-5 hours. The cells were centrifuged 8,000×g for 1 minute. The pelleted Agrobacterium cells were resuspended in 100 μl of the supernatant and plated on a Petri dish containing LB medium supplemented with 40 m/ml kanamycin and 25 m/ml rifampicin. Following a two-day incubation at 28° C., single clones of appropriate size were inoculated into YEB liquid medium and shake-cultured at 28° C. until the OD₆₀₀ reached 0.4-0.6. The resulted culture can be used for transformation and co-culture of the rice.

9. Agrobacterium-Mediated Transformation of Expression Vector in Rice.

9.1 Induction and subculture of calluses. Rice seeds, either mature or immature, were surface-sterilized by 70% alcohol for 1.5 minutes followed by deep sterilization by shaking in a solution of 20% sodium hypochlorite containing a drop of Tween-20 for 45 minutes at 28° C. The seeds were thoroughly washed in ddH₂O until the washwater was clear and then dark-cultured on inducing medium at 25° C. for about 3 weeks. Induced calluses were transferred to fresh inducing medium for the first subculturing. Subculturing was repeated every three to four weeks. Following two subculturings, embryogenic calluses were crisp, brilliant yellow, and 3-5 mm long, were used in the next step of co-culture.

TABLE 4
Genetic Transformation Media

| | Media | | | | |
|---|---|---|---|---|---|
| Composition | | Co- | | | |
| (mg/ml) | Induce | culture | Selection | Different. | Rooting |
| NB | 1x | 1x | 1x | 1x | ½x |
| Casein hydrolysate | 300 | 300 | 300 | 300 | — |
| Proline | 500 | 500 | 500 | 500 | — |
| Glutamine | 500 | 500 | 500 | 500 | — |
| 2,4- | 2.0 | 2.0 | 2.0 | — | — |
| Dichlorophenoxy- | | | | | |
| acetate | | | | | |
| Sucrose (%) | 3 | 3 | 3 | 3 | 3 |
| Agar (%) | 0.8 | 0.8 | 0.8 | 0.8 | 0.8 |
| Acetosyringone | — | 39.6 | — | — | — |
| Hygromycin | — | — | 40 | 25 | — |
| Timentin | — | — | 300 | — | — |
| (Ticarcillin- | | | | | |
| Potassium | | | | | |
| Clavulanate) | | | | | |
| α-Naphthaleneacetic | — | — | — | — | 0.5 |
| acid | | | | | |
| Benzylaminopurine | — | — | — | 2.0 | — |
| Kinetin | — | — | — | 0.5 | — |
| Hygromycin | — | — | 50 | 25 | — |
| Cefatoxime | — | — | 250 | — | — |
| pH | 5.8 | 5.8 | 5.8 | 5.8 | 5.8 |

### NB Media:

2830 mg/L KNO₃; 463 mg/L (NH₄)₂SO₄; 400 mg/L KH₂PO₄;
185 mg/L MgSO₄.7H₂O; 166 mg/L CaCl₂.2H₂O, 27.8 mg/L FeSO₄.7H₂O;
37. 5 mg/L Na₂EDTA; 10 mg/L MnSO₄.4H₂O; 3 mg/L H₃BO₃;
2 mg/L ZnSO₄.7H₂O; 0.25 mg/L Na₂MoO₄.2H₂O; 0.025 mg/L CuSO₄.5H₂O;
0.025 mg/L CoCl₂.6H₂O; 0.75 mg/L KI;
10 mg/L Vitamin B1 (Thiamine hydrochloride);
1 mg/L Vitamin B6 (Pyridoxine hydrochloride);
1 mg/L nicotinic acid; 100 mg/L inositol.

10.2 Co-culture of calluses and Agrobacterium tumefaciens. Well-growing embryogenic rice calluses were placed in a sterilized Petri dish, and fresh Agrobacterium (OD₆₀₀ 0.4-0.6) was added with shaking shaking After a one-hour co-culture, calluses were removed and residual medium blotted on sterilized filter. Calluses were transferred to co-culture medium and co-cultured at 25° C. in dark for 2-3 days.

10.3 Removal of Agrobacterium. After a 2-3 day co-culture, Agrobacterium plaques were observed on the calluses. Calluses were picked out, put into a sterilized flask, and extensively washed by shaking in sterilized water containing 250 mg/L carbenicillin, until the filar thallus was no longer visible in the wash water. Calluses continued to soak in the wash water for 1 hour to allow adhered Agrobacterium to desorb from the calluses. Calluses were further shaken at 120 rpm for 2 hours at 25° C. in sterilized water containing 500 mg/L carbenicillin. Calluses were removed and blotted on sterilized filter paper.

10.4 Selection of resistant calluses. Calluses were transferred to selecting medium and dark-cultured at 25° C. with periodic examination of Agrobacterium contamination. Subculturing was repeated every two weeks. After 4-8 weeks, most of calluses were dead as indicated by a browned appearance. Tuberculate (resistant) calluses, that grew out of the surface of browned calluses, were subcultured on selecting medium. Fully-grown calluses were transferred to differentiating medium.

10.5 Differentiating culture of the resistant calluses. Antibiotic resistant calluses were transferred to differentiating medium and dark-cultured at 26° C. for one week followed by light-culture at 25° C. (light 16 h/dark 8 h).

10.6 Regeneration and seedling transplantation of the transgenic plants. Calluses, transferred to differentiating medium, greened after two weeks and sprouted and rooted after three weeks. When regenerated seedlings grew to 2-3 cm, they were transferred to rooting medium and light-cultured. When they grew to 7-10 cm, they were trained in greenhouse for 5-7 days in open flasks. Seedlings were removed from flasks when they were strong and the medium was wash off from the root. Seedling were potted in the greenhouse at high humidity to ensure their rate.

11. PCR identification of the transgenic plants.

DNA from leaves of regenerated plants was extracted and used as templates for PCR identification as described above using the conditions of Table 5.

TABLE 5
Primers and Amplification Conditions

| | | Size | | Amplification |
|---|---|---|---|---|
| Gene | Primer Sequences | (bp) | Gel% | procedure |
| GUS gene | 5pGUS: | 522 | 1.4% | 94° C. 5 mins. |
| | 5′-TCTACACCACGCCGAACACCT-3′ | | | 94° C. 1 mins. |
| | 3pGUS: | | | 58° C. 50 sec. |
| | 5′-GCCGACAGCAGCAGTTTCATC-3′ | | | 72° C. 30 sec. |
| | | | | 28 Cycles |
| | | | | 72° C. 10 mins. |
| 35S | 5p35S: | 195 | 2.0% | 94° C. 9 mins. |
| promoter | 5′-TCCTACAAATGCCATCATTGC-3′ | | | 94° C. 30 sec. |
| | 3p35S : | | | 62° C. 30 sec. |
| | 5′-TAGTGGGATTGTGCGTCATCC-3′ | | | 72° C. 30 sec. |
| | | | | 35 Cycles |
| | | | | 72° C. 7 mins. |

12. Histochemical Staining for Localization of GUS.

12.1. Transplantation of positive transgenic plants (T₀). Positive, transgenic plants, confirmed by PCR, were transplanted to fields. Histochemical staining for GUS activity was performed on 15 sample seedling plants on root, stem, leaf, and organ tissues for each sample. Histochemical staining for GUS activity was also performed on samples of immature (16 days after anthesis) and mature (30 days after anthesis) endosperm of plants. Non-transgenic rice was used as negative control.

12.2. GUS staining protocol. Samples were incubated in reaction solution (Table 6) at 37° C. for 2-6 hours. Chlorophyll was removed from chlorenchyma by incubation in 70% alcohol at room temperature for 5 hours. This step was repeated until all the chlorophyll was removed. Reaction solution is prepared as follows: X-Gluc (5-bromo-4-chloro-3-indolyl-beta-D-glucuronic acid cyclohexylammonium salt) was dissolved in N,N-dimethylformamide with stirring, and then 0.1 mol/L phosphate buffer, 5 mmol/L potassium ferricyanide and 5 mmol/L potassium ferrocyanide were added into the X-Gluc solution with stirring. Finally, Triton X-100 was added. This solution should be prepared shortly before use.

TABLE 6
Reaction solution: X-Gluc solution

| Reaction components | amount | |
|---|---|---|
| N,N-dimethylformamide | 1-2 | drops |
| X-Gluc | 1 | mg |
| 0.1 mol/L phosphate buffer (pH 0.7) | 980 | μl |
| 5 mmol/L potassium ferricyanide | 10 | μl |
| 5 mmol/L potassium ferrocyanide | 1 | ddw |
| Triton X-100 | 1 | μl |
| Sterilized water was supplemented to | 1 | ml |

We claim:

1. An isolated promoter comprising an isolated polynucleotide having a sequence selected from the group consisting of SEQ ID NO: 1 and a sequence that hybridizes to SEQ ID NO: 1 under conditions of defined stringency.

2. The polynucleotide according to claim 1, wherein said conditions are low stringency.

3. The polynucleotide according to claim 1, wherein said conditions are high stringency.

4. The promoter of claim 1, wherein said promoter drives the transcription of an operably-linked gene in a plant non-endosperm tissue.

5. An expression cassette comprising:
a promoter comprising an isolated polynucleotide having a sequence selected from the group consisting of SEQ ID NO: 1 and a sequence that hybridizes to SEQ ID NO: 1 under conditions of defined stringency; and,
an encoding region operably linked to said promoter, wherein said promoter shows transcriptional activity in non-endosperm plant tissues of plants transformed by said expression cassette.

6. The expression cassette according to claim 5, wherein said polynucleotide sequence is inserted into the expression cassette in sense direction.

7. The expression cassette according to claim 5, wherein said polynucleotide sequence is inserted into the expression cassette in anti-sense direction.

8. The expression cassette according to claim 5, wherein said promoter drives the encoding region to be transcribed and expressed in the non-endosperm tissues of the plants transformed by the expression cassette.

9. An expression vector comprising:
An expression cassette further comprising:
a promoter comprising an isolated polynucleotide having a sequence selected from the group consisting of SEQ ID NO: 1 and a sequence that hybridizes to SEQ ID NO: 1 under conditions of defined stringency; and,
an encoding region operably linked to said promoter, wherein said promoter shows transcriptional activity in non-endosperm plant tissues of plants transformed by said expression cassette.

10. The expression vector according to claim 9, wherein said vector comprises a nucleic acid construct selected from the group consisting of plasmids, cosmids, phages, and binary vectors.

11. The expression vector according to claim 10, wherein said binary vector is an Agrobacterium binary vector.

12. A primer comprising a nucleic acid molecule selected from the group consisting of SEQ ID NO: OsTSP I forward primer and SEQ ID NO: OsTSP I reverse primer.

13. A primer comprising a nucleic acid molecule that drives the PCR amplification of SEQ ID NO: OsTSP I.

14. The expression cassette according to claim 5, wherein said cassette is stably incorporated into a plant genome.

15. The expression vector according to claim 9, wherein said vector is stably incorporated into a plant genome.

16. A plant comprising at least one plant cell stably-transformed by an exogenous promoter comprising an exogenous polynucleotide having a sequence selected from the group consisting of SEQ ID NO: 1 and a sequence that hybridizes to SEQ ID NO: 1 under conditions of defined stringency.

17. A plant comprising at least one plant cell stably-transformed by an expression cassette further comprising:
a promoter comprising an exogenous polynucleotide having a sequence selected from the group consisting of SEQ ID NO: 1 and a sequence that hybridizes to SEQ ID NO: 1 under conditions of defined stringency; and,
an encoding region operably linked to said promoter, wherein said promoter shows transcriptional activity in non-endosperm plant tissues of plants transformed by said expression cassette.

18. A plant comprising at least one plant cell stably-transformed by n expression vector comprising:
an expression cassette further comprising:
a promoter comprising an isolated polynucleotide having a sequence selected from the group consisting of SEQ ID NO: 1 and a sequence that hybridizes to SEQ ID NO: 1 under conditions of defined stringency; and,
an encoding region operably linked to said promoter, wherein said promoter shows transcriptional activity in non-endosperm plant tissues of plants transformed by said expression cassette.

19. A plant seed comprising the promoter of claim 1.

20. A plant seed comprising the expression cassette of claim 5.

21. A plant seed comprising the expression vector of claim 9.

22. A transgenic plant cell, tissue, organ, or seed comprising an expression cassette according to claim 5, wherein said encoding region encodes an anti-insect protein, an anti-bacterial protein, an anti-fungal protein, an anti-viral protein (or polypeptide product or RNA molecule), an anti-nematode protein, an anti- herbicide protein, or an selectable marker protein.


SEQUENCE LISTING (8 sequences)

SEQ ID NO: 1 - DNA, 1785 bp, Oryza sativa
  promoter (1)..(1785): n = a or g or c or t
gtccgtttccgttcgttaattggtactactacctacgcgtagcgtgttgctccctaaacaactcccagatcaggcaaaggaagcatcgtctcgtctgcacgtactctaccaagaaaatgatcagcgccatggaggccagaacatgcacacatgcggtgcgacccctcacatggggcaacagggcatgctgcaaactgaagagtcgaagaccacggttccctccccatggataaaagatctggtcttttcagttatcagtgtccggcatatgtatggggatcaagtggtgggggcaaaaaaaaaaaaaaaccagtggcatgatcgggcacagctcgcgtcggaacaaggcatcgtgtcacatggaagaaacccatcgcttgttttatggaccgcgcggcgcgcgcgcgcatgcggaccgcgcggtgactcctgtccctgtgcaggttgacggcgagcacatgcctagctacgtcgtggtagccccctgcaacgtcccacgtacgcgcatgcaaattgcagcatcacgattggtctggaattgtacatttgtactctctgcaccagggaaaaattttgtccagattgcaggggagagatacggttggtgctgtgcgccgtgctgcgaatactgcgtccagtcaggcagactcgagctcggtcggtcacacgaacaggcgtgcatgcatgaggcacgcaggccggtcaatcgccttgcacgcacacacacatcctcgggtcgatctggccatctgggtcgcgtgctggtttgggtggaatcgagtttctagttttgtcttgcgttacgatttcccctgttcgggtgtgttgtaatcttgttgcggactcgcggagtcgcggtatatactcggtacatgtatattgcaatttgcgaggggggtttgggtttcctcgcgcaatcaagtgcgtatatacttaagacgcgcgcacacatgggcgccatgtgtcggttgagtatccttgtcagggtttgatccagatacatgatgctgtccggccttccagcctacaacatgatcctggaaatgatgtgatgacatgagtacacgatctgaacctacgatttccatgattaaactgagcttcacaacctcgggccacaagaattttcacgtgaagccgttcgaattgcatgcgagtatgcaacttactcctacatcacgaaaaatggtccataccgcaaagggaaaaaagaaagttccaagtgccatggtaaccagctcactcagtgacaaaagtggtgaaagattcctaaacaccggcacgccacagcgtccagccggtgcccggttgtgcgactacgatgcttgtcccctcgcaaaatcccatgatgaacgctaaccattaaccaacttgattacatacggcggcatctgtgttatcacgggaaccgcagaggcatacgtaaccgacgaaaaaaacgcggacgagatggcgaaactgcccctcgtcgtgcaccgcctcaccgggccgaaagccagtcgcgtgcgcgtgcagagagacggcgcgccgcacgtactgtacacgagccggtgcgcgcggtaggaaacggaagcggatcagggggccatgtgaccgcacgcagggcgtgtctcctacgaggccacgagggcagagggagcccatcatccgctcagccgaatcgccgatcggggacacgcgtacggcggaagatcccgtggcatttcgtggtagtaatcgaccaaccctaggcccgtttcgccggcagcttggtctataagttgctgctctgccctgctcggcctc

SEQ ID NO: 2 - DNA, 33 bp, Artificial Sequence
  Forward primer for amplification of promoter OsTSP I (containing EcoRI restriction site and protective bases)
gatcatcgaattcgtccgtttccgttcgttaat

SEQ ID NO: 3 - DNA, 33 bp, Artificial Sequence
  Reverse primer for amplification of promoter OsTSP I (containing BamHI restriction site and protective bases)
agtcagtggatccgaggccgagcagggcagagc

SEQ ID NO: 4 - DNA, 1811 bp, Oryza sativa
  EcoR1 Site (8)..(13)
  BamH1 Site (1799)..(1804)
gatcatcgaattcgtccgtttccgttcgttaattggtactactacctacgcgtagcgtgttgctccctaaacaactcccagatcaggcaaaggaagcatcgtctcgtctgcacgtactctaccaagaaaatgatcagcgccatggaggccagaacatgcacacatgcggtgcgacccctcacatggggcaacagggcatgctgcaaactgaagagtcgaagaccacggttccctccccatggataaaagatctggtcttttcagttatcagtgtccggcatatgtatggggatcaagtggtgggggcaaaaaaaaaaaaaaaccagtggcatgatcgggcacagctcgcgtcggaacaaggcatcgtgtcacatggaagaaacccatcgcttgttttatggaccgcgcggcgcgcgcgcgcatgcggaccgcgcggtgactcctgtccctgtgcaggttgacggcgagcacatgcctagctacgtcgtggtagccccctgcaacgtcccacgtacgcgcatgcaaattgcagcatcacgattggtctggaattgtacatttgtactctctgcaccagggaaaaattttgtccagattgcaggggagagatacggttggtgctgtgcgccgtgctgcgaatactgcgtccagtcaggcagactcgagctcggtcggtcacacgaacaggcgtgcatgcatgaggcacgcaggccggtcaatcgccttgcacgcacacacacatcctcgggtcgatctggccatctgggtcgcgtgctggtttgggtggaatcgagtttctagttttgtcttgcgttacgatttcccctgttcgggtgtgttgtaatcttgttgcggactcgcggagtcgcggtatatactcggtacatgtatattgcaatttgcgaggggggtttgggtttcctcgcgcaatcaagtgcgtatatacttaagacgcgcgcacacatgggcgccatgtgtcggttgagtatccttgtcagggtttgatccagatacatgatgctgtccggccttccagcctacaacatgatcctggaaatgatgtgatgacatgatgacacgatctgaacctacgatttccatgattaaactgagcttcacaacctcgggccacaagaattttcacgtgaagccgttcgaattgcatgcgagtatgcaacttactcctacatcacgaaaaatggtccataccgcaaagggaaaaaagaaagttccaagtgccatggtaaccagctcactcagtgacaaaagtggtgaaagattcctaaacaccggcacgccacagcgtccagccggtgcccggttgtgcgactacgatgcttgtcccctcgcaaaatcccatgatgaacgctaaccattaaccaacttgattacatacggcggcatctgtgttatcacgggaaccgcagaggcatacgtaaccgacgaaaaaaacgcggacgagatggcgaaactgcccctcgtcgtgcaccgcctcaccgggccgaaagccagtcgcgtgcgcgtgcagagagacggcgcgccgcacgtactgtacacgagccggtgcgcgcggtaggaaacggaagcggatcagggggccatgtgaccgcacgcagggcgtgtctcctacgaggccacgagggcagagggagcccatcatccgctcagccgaatcgccgatcggggacacgcgtacggcggaagatcccgtggcatttcgtggtagtaatcgaccaaccctaggcccgtttcgccggcagcttggtctataagttgctgctctgccctgctcggcctcggatccactgact

SEQ ID NO: 5 - DNA, 19 bp, Artificial Sequence
  Forward promoter used to amplify GI21104672
gaacagtccagcagcgtaa

SEQ ID NO: 6 - DNA, 19 bp, Artificial Sequence
  Reverse primer gene GI21104672
ccacagcccaccatcatac

SEQ ID NO: 7 - DNA, 20 bp, Artificial Sequence
  Forward primer actin gene
tatggtcaaggctgggttcg

SEQ ID NO: 8 - DNA, 20 bp, Artificial Sequence
  Reverse primer actin gene
ccatgctcgatggggtactt